(19) United States
(12) Patent Application Publication
(10) Pub. No.: US 2021/0307807 A1
(43) Pub. Date: Oct. 7, 2021

(54) MULTI-MODALITY FORCEPS

(71) Applicant: Covidien LP, Mansfield, MA (US)

(72) Inventors: Craig V. Krastins, Arvada, CO (US); Grant T. Sims, Boulder, CO (US); Daniel W. Mercier, Erie, CO (US); Jennifer L. Rich, Parker, CO (US); Kelley D. Goodman, Erie, CO (US)

(21) Appl. No.: 17/220,322

(22) Filed: Apr. 1, 2021

Related U.S. Application Data
(60) Provisional application No. 63/004,247, filed on Apr. 2, 2020.

(51) Int. Cl. A61B 18/08; A61B 18/14; A61B 18/12

(52) U.S. Cl. CPC A61B 18/085; A61B 18/1442; A61B 2018/00595; A61B 2018/1253; A61B 2018/126; A61B 18/1206

(57) ABSTRACT

A surgical system includes a forceps having an end effector at a distal end thereof that includes first and second opposing jaw members electrically conductive plates the jaw members pivotable relative to one another between an open position and a closed position. A generator is included that is configured to produce multiple modalities of electrical energy upon activation thereof. A first cable connects to the generator, the first cable including leads disposed therein configured to carry energy to the plates. A first switch is disposed on the forceps. A second cable connects to the electrical generator and to a second switch. Activation of the first switch activates the generator to transmit energy having a first modality through the first cable and to the plates and activation of the second switch activates the generator to transmit electrical energy having a second modality through the first cable to the opposing electrical plates.

### CROSS REFERENCE TO RELATED APPLICATION

This application claims the benefit of U.S. Provisional Application No. 63/004,247, filed on Apr. 2, 2020.

### BACKGROUND

### Technical Field

The present disclosure relates to surgical instruments and, more particularly, to a surgical forceps having multiple energy modalities for facilitating surgical procedures.

### Description of Related Art

A surgical forceps is a plier-like instrument which relies on mechanical action between its jaws to grasp tissue. Electrosurgical forceps utilize both mechanical clamping action and electrical energy to treat tissue, e.g., coagulate, cauterize, and/or seal tissue.

Typically, once tissue is treated, the surgeon has to accurately sever the treated tissue. Accordingly, many electrosurgical forceps have been designed which incorporate a knife configured to effectively sever tissue after treating the tissue.

Various types of surgical forceps utilize different types of energy modalities to coagulate, cauterize, transect or seal vessels. For example, a range of forceps utilize a Ligasure® sealing algorithm designed to seal small vessels using a combination of compression pressure, gap control and bipolar radiofrequency (RF) energy to shrink tissue collagen and elastin in the vessel wall. When it is desirous to treat vessels or tissue in a conventional bipolar manner (e.g., to cauterize or transect tissue to control bleeding), a bipolar forceps is substituted for the Ligasure® forceps. During head and neck surgery, both energy devices are constantly swapped depending on the particular surgical need. A single device with the ability to function with multiple energy modalities would be advantageous during these type of surgical procedures.

### SUMMARY

As used herein, the term “distal” refers to the portion that is being described which is further from a user, while the term “proximal” refers to the portion that is being described which is closer to a user.

Provided in accordance with one aspect of the present disclosure, a forceps having one or more shafts is configured to support an end effector assembly at a distal end thereof. The end effector includes first and second opposing jaw members each having an electrically conductive plate associated therewith configured to communicate electrosurgical energy therebetween. One or both of the first and second jaw members is pivotable relative to the other about a pivot such that the jaw members are selectively movable between an open position wherein the jaw members are spaced relative to one another and a closed position for grasping tissue therebetween. An electrical generator is included and is configured to produce multiple modalities of electrical energy upon activation thereof.

A first electrical cable is operably connected at one end to a first port defined in the electrical generator and at an opposite end to the forceps, the first electrical cable includes electrical leads disposed therein configured to carry electrical energy to opposing electrical plates of the jaw members. A first switch is disposed on the forceps and is disposed in electrical communication with one or both of the electrical leads. A second cable is operably connected at one end to a second port defined in the electrical generator and at an opposite end to a second switch. Activation of the first switch activates the electrical generator to transmit electrical energy having a first modality through the first electrical cable and to the opposing electrical plates of the jaw members and activation of the second switch activates the electrical generator to transmit electrical energy having a second modality through the first electrical cable to the opposing electrical plates of the jaw members.

In aspects according to the present disclosure, the first modality of electrical energy includes a sealing energy delivery algorithm. In other aspects according to the present disclosure, the second modality of electrical energy includes a bipolar energy delivery algorithm.

In aspects according to the present disclosure, the first switch includes an activation switch disposed on one shaft of the forceps. In aspects according to the present disclosure, the first activation switch is an in-line activation switch that is selectively activatable when the jaw members are moved from the open position to the closed position.

In aspects according to the present disclosure, the second switch includes a footswitch remotely disposed relative to the generator. In other aspects according to the present disclosure, the first activation switch has priority over the second activation switch when activated. In yet other aspects according to the present disclosure, substantially simultaneous activation of the first activation switch and the second activation switch defaults the generator to deliver a third energy modality to one or both of the electrically conductive plates of the jaw members. In still other aspects according to the present disclosure, the third modality of electrical energy includes a monopolar energy delivery algorithm.

Provided in accordance with one aspect of the present disclosure, a forceps includes one or more shafts configured to support an end effector assembly at a distal end thereof. The end effector includes first and second opposing jaw members each having an electrically conductive plate associated therewith configured to communicate electrosurgical energy therebetween. One or both of the jaw members is pivotable relative to the other about a pivot such that the jaw members are selectively movable between an open position wherein the jaw members are spaced relative to one another and a closed position for grasping tissue therebetween.

An electrical cable is adapted to operably connect to a first port defined in an electrical generator, the electrical cable including electrical leads disposed therein configured to carry electrical energy to opposing electrical plates of the jaw members. A first switch is disposed on the forceps and is disposed in electrical communication with one or both of the electrical leads. Activation of the first switch activates the electrical generator to transmit electrical energy having a first modality through the electrical cable and to the opposing electrical plates of the jaw members and activation of a second switch operably connected to and remotely disposed from the forceps activates the electrical generator to transmit electrical energy having a second modality through the electrical cable to the opposing electrical plates of the jaw members.

In aspects according to the present disclosure, the first modality of electrical energy includes a sealing energy delivery algorithm. In other aspects according to the present disclosure, the second modality of electrical energy includes a bipolar energy delivery algorithm.

In aspects according to the present disclosure, the first activation switch is an in-line activation switch that is selectively activatable when the jaw members are moved from the open position to the closed position.

In aspects according to the present disclosure, the jaw members are tapered from a proximal end thereof to a distal end thereof. In other aspects according to the present disclosure, when disposed in the closed position, the distal ends of each jaw member combine to form a low profile tip to facilitate fine tissue dissection and cautery.

### BRIEF DESCRIPTION OF THE DRAWINGS

Various aspects of the present disclosure are described herein with reference to the drawings wherein like reference numerals identify similar or identical elements:

FIG. 1 is a side, perspective view of a forceps including opposing shaft members and an end effector assembly disposed at a distal end thereof according to an aspect of the present disclosure;

FIG. 2 is side view of the forceps of FIG. 1;

FIG. 3 is schematic diagram of a multi-modality surgical system including the forceps of FIG. 1 coupled to an electrosurgical generator and a foot switch connected the generator;

FIG. 4 is an enlarged view of a distal end of the forceps of FIG. 1 with a schematic representation of an electrical diagram for use with the multi-modality system;

FIGS. 5A-5B are top view of the forceps of FIG. 1 superimposed over a convention a bayonet forceps; and

FIG. 6 is a side perspective view of an endoscopic surgical forceps for use with the multi-modality surgical system.

### DETAILED DESCRIPTION

Throughout the description, like reference numerals and letters indicate corresponding structure throughout the several views. Also, any particular feature(s) of a particular exemplary embodiment may be equally applied to any other exemplary embodiment(s) of this specification as suitable. In other words, features between the various exemplary embodiments described herein are interchangeable as suitable, and not exclusive.

Embodiments of the disclosure include systems, devices, and methods to control tissue temperature at a tissue treatment site during an electrosurgical procedure, as well as shrinking, coagulating, cutting, and sealing tissue against blood and other fluid loss, for example, by shrinking the lumens of blood vessels (e.g., arteries or veins). In some embodiments, the devices may be configured, due to the narrow electrode size, to fit through a trocar down to a size as small as 5 mm.

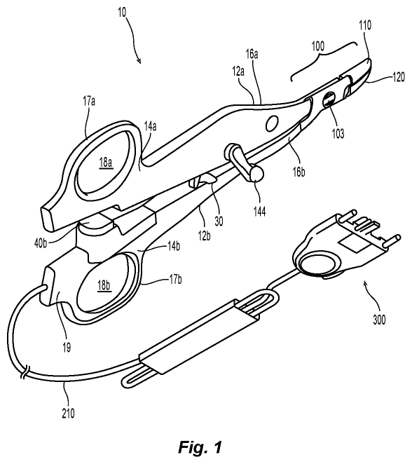

Referring now to FIG. 1, an open forceps 10 contemplated for use in connection with traditional open surgical procedures is shown. For the purposes herein, either an open instrument, e.g., forceps 10, or an endoscopic instrument (not shown) may be utilized in accordance with the present disclosure. Obviously, different electrical and mechanical connections and considerations apply to each particular type of instrument; however, the novel aspects with respect to the end effector assembly and its operating characteristics remain generally consistent with respect to both the open and endoscopic configurations.

With continued reference to FIG. 1, forceps 10 includes two elongated shafts 12a and 12b, each having a proximal end 14a and 14b, and a distal end 16a and 16b, respectively. Forceps 10 further includes an end effector assembly 100 attached to distal ends 16a and 16b of shafts 12a and 12b, respectively. End effector assembly 100 includes a pair of opposing jaw members 110 and 120 that are pivotably connected about a pivot 103. Each shaft 12a and 12b includes a handle 17a and 17b disposed at the proximal end 14a and 14b thereof. Each handle 17a and 17b defines a finger hole 18a and 18b therethrough for receiving a finger of the user. Finger holes 18a and 18b facilitate movement of the shaft members 12a and 12b relative to one another between a spaced-apart position and an approximated position, which, in turn, pivot jaw members 110 and 120 from an open position, wherein the jaw members 110 and 120 are disposed in spaced-apart relation relative to one another, to a closed position, wherein the jaw members 110 and 120 cooperate to grasp tissue therebetween.

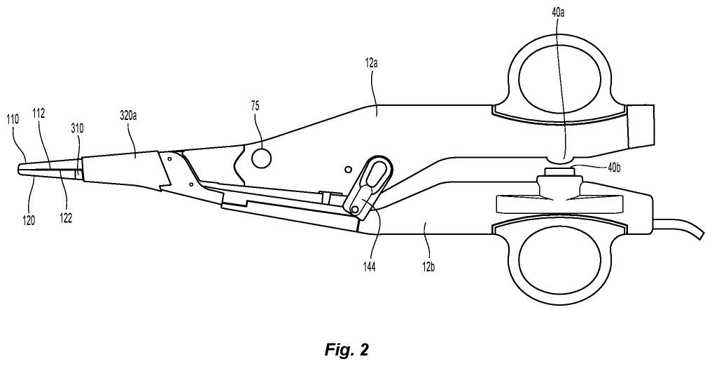

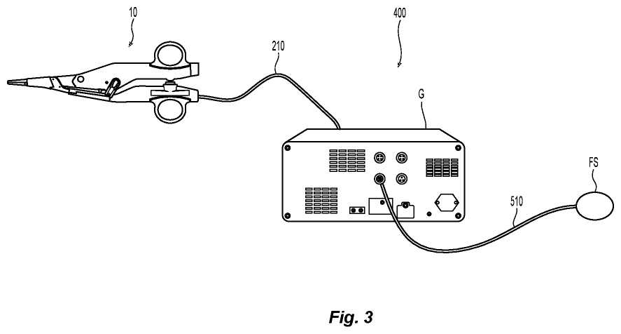

Continuing with reference to FIG. 1, one of the shafts, e.g., shaft 12b, includes a proximal shaft connector 19 that is designed to connect the forceps 10 to a source of electrosurgical energy such as an electrosurgical generator G (FIG. 3). Proximal shaft connector 19 secures an electrosurgical cable 210 to forceps 10 such that the user may selectively apply electrosurgical energy to electrically-conductive plates 112 and 122 (see FIG. 2) of jaw members 110 and 120, respectively.

More specifically, cable 210 includes a plurality of wires (not shown) extending therethrough that has sufficient length to extend through one of the shaft members, e.g., shaft member 12b, in order to provide a first modality of electrical energy to the conductive plates 112, 122 of jaw members 110, 120, respectively, of end effector assembly 100, e.g., upon activation of activation switch 40b (See FIGS. 1 and 2) or, as explained in more detail below, upon activation of a second switch, e.g., footswitch FS (FIG. 3), in order to provide a second modality of electrical energy to one or both conductive plates 112, 122. Other types activation switches are also contemplated, e.g., finger switch, toggle switch, foot switch, etc. and may be configured for this purpose. Cable 210 operably connects to generator G via plug 300.

Activation switch 40b is disposed at proximal end 14b of shaft member 12b and extends therefrom towards shaft member 12a. A corresponding surface 40a is defined along shaft member 12a toward proximal end 14a thereof and is configured to actuate activation switch 40b. More specifically, upon approximation of shaft members 12a, 12b, e.g., when jaw members 110, 120 are moved to the closed position, activation switch 40b is moved into contact with, or in close proximity of surface 40a. Upon further approximation of shaft members 12a, 12b, e.g., upon application of a pre-determined closure force to jaw members 110, 120, activation switch 40b is advanced further into surface 40a to depress activation switch 40b. Activation switch 40b controls the supply of a first modality of electrosurgical energy to jaw members 110, 120 such that, upon depression of activation switch 40b, electrosurgical energy is supplied to conductive surface 112 and/or conductive surface 122 of jaw members 110, 120, respectively, to seal tissue grasped therebetween. The first modality of electrical energy may be energy supplied through a proprietary Ligasure® sealing algorithm LS owned by Covidien, LP (Medtronic). The switch 40b may be disposed on either shaft 12a, 12b.

Referring now to FIGS. 2 and 3, in conjunction with FIG. 1, forceps 10 may further include a knife assembly (not shown) disposed within one of the shaft members, e.g., shaft member 12a and a knife channel (not shown) defined within one or both of jaw members 110, 120, respectively, to permit reciprocation of a knife (not shown) therethrough. Knife assembly includes a rotatable trigger 144 coupled thereto that is rotatable about a pivot for advancing the knife from a retracted position within shaft member 12a, to an extended position wherein the knife extends into knife channels to divide tissue grasped between jaw members 110, 120. In other words, axial rotation of trigger 144 effects longitudinal translation of knife. Other trigger assemblies are also contemplated.

Each jaw member 110, 120 of end effector assembly 100 may include a jaw frame having a proximal flange extending proximally therefrom that are engagable with one another to permit pivoting of jaw members 110, 120 relative to one another about a pivot 103 between the open position and the closed position upon movement of shaft members 12a, 12b relative to one another between the spaced-apart and approximated or closed positions. Proximal flanges of jaw members 110, 120 also connect jaw members 110, 120 to the respective shaft members 12b, 12a thereof, e.g., via welding, crimping or the like.

Jaw members 110, 120 may each further include an insulator (not shown) that is configured to receive a respective electrically-conductive tissue plate 112, 122, thereon and that is configured to electrically isolate the conductive plates 112, 122 from the remaining components of the respective jaw members 110, 120 (FIG. 2). Conductive plates 112, 122 are disposed in opposed relation relative to one another such that, upon movement of jaw members 110, 120 to the closed position, tissue is grasped between conductive plates 112, 122, respectively, thereof. Accordingly, in use, one or more modalities of electrosurgical energy may be supplied to one or both of conductive plates 112, 122 and conducted through tissue to treat tissue grasped therebetween. Knife may be advanced through knife channels of jaw members 110, 120 to cut tissue before, during or after treatment.

Turning to FIG. 3, a schematic representation of a surgical system 400 is shown and includes forceps 10, generator G and footswitch FS. In use, the forceps 10 connects to the generator G via plug 300 (See FIG. 1). Activation of switch 40b of the forceps 10 provides electrical energy to the conducive plates 112, 122 utilizing a proprietary Ligasure® sealing algorithm LS to seal tissue disposed between the jaw members 110, 120. The user squeezes handles 17a, 17b which, in turn, approximates the jaw members 110, 120. If it is desirous to seal tissue, the user fully approximates the handles 17a, 17b to activate activation switch 40b disposed therebetween. Once sealed, the user may actuate the knife assembly to cut the tissue disposed between the jaw members 110, 120.

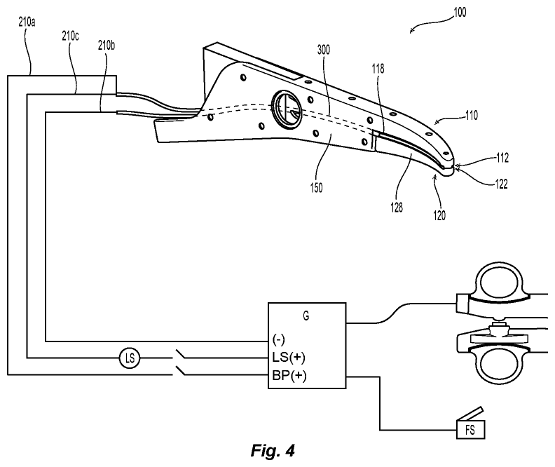

As mentioned above, a footswitch FS is operably coupled to the generator G via cable 510. Upon actuation of the footswitch FS, electrical energy is transmitted to the conductive plates 112, 122 to treat tissue in a standard bipolar manner, e.g., for use with cauterizing tissue. The footswitch FS does not supply the necessary electrical energy to the tissue, but rather, sends a control signal to the generator G to apply standard or known electrical, bipolar energy across leads 210a, 210b to treat tissue (FIG. 4). Similarly, if the activation switch 40b is actuated upon full approximation of the jaw members 110, 120, a control signal is sent to the generator to apply electrical energy across the leads 210c, 210b utilizing the Ligasure® sealing algorithm LS.

Many iterations of the Ligasure® sealing algorithm LS have been developed over the years and, as such, when using the term Ligasure® sealing algorithm LS, all of these various iterations are envisioned. Details relating to some of the iterations of the Ligasure® sealing algorithm LS are disclosed in U.S. Pat. Nos. 8,920,421, 8,216,223, 6,398,779, 7,901,400, 7,972,328 the entire contents of each of which being incorporated by reference herein

When switch 40b is depressed, the generator recognize a voltage drop across leads 210b and 210c which initiates activation of the generator G to supply a first electrical potential to jaw member 110 and a second electrical potential to jaw member 120 in a first energy modality, e.g., energy delivered pursuant to the Ligasure® algorithm LS. In this fashion, switch 40b acts more like a control circuit and is protected or removed from the actual current loop which supplies electrical energy to the jaw members 110 and 120. This reduces the chances of electrical failure of the switch 40b due to high current loads during activation. As mentioned above, footswitch FS also operates in a similar manner, i.e., upon activation of the footswitch FS, the generator recognizes a voltage drop across the leads 210a, 210b which, in turn, signals the generator G to initiate electrosurgical activation of bipolar energy the jaw members 110 and 120.

Various safety features are also envisioned to control the energy delivery to the jaw member 110, 120. For example, internal software in the generator G may prioritize the Ligasure® activation switch 40b and the bipolar footswitch FS, e.g., if the Ligasure® activation switch 40b is activated it will take precedence over the footswitch FS and energy will be delivered pursuant to the Ligasure® algorithm LS. In embodiments according to the present disclosure, if footswitch FS is activated and the user actuates switch 40b, energy will be switched to deliver energy utilizing the first energy modality or pursuant to the Ligasure® algorithm LS. In other embodiments according to the present disclosure, if switch 40b is actuated and then footswitch FS is depressed, energy delivery is continued utilizing the first energy modality or pursuant to the Ligasure® algorithm LS. In other embodiments, the internal software may simply elect to continue with the algorithm associated with the initial activation switch, e.g., activation switch 40b or footswitch FS. In yet other embodiments, if both switches e.g., activation switch 40b and footswitch FS, are activated at the same time or substantially at the same time, a third algorithm may be employed.

In other embodiments according to the present disclosure, if both switches 40b and footswitch FS are depressed simultaneously or substantially simultaneously, the energy delivery may again default to the first energy modality pursuant to the Ligasure® algorithm LS or a third energy modality may be introduced, e.g., a monopolar energy modality wherein only one electrode is activated a return path is established through the patient and to a patient return pad (Not shown). Alternatively, if both switches 40b and footswitch FS are depressed simultaneously or substantially simultaneously, the generator G may simply pause for a recalibration or a possible regrasp, put the forceps in a “timeout” or delay mode, trigger an alarm or, possibly, provide a third energy modality with a third algorithm.

Various tactile, audible and/or visual displays or alarms may be utilized to inform or confirm to the user that the proper or desired energy modality is being utilized. In other embodiments, alarms may be utilized to address concerns relating to energy delivery or switch priority concerns.

During head and neck surgeries it is typical for a surgeon to switch between forceps employing a first modality of energy to the opposing conductive plates 112, 122 pursuant to the Ligasure® algorithm LS and bayonet style forceps or jeweler-style forceps (or Adson forceps) employing a second modality of energy to the opposing conductive plates 112, 122 pursuant to more standard bipolar algorithms. Forceps 10 enables the surgeon to easily switch back and forth between energy modalities by actuating the two switches 40b and footswitch FS at different times according to a desired energy modality. Or, as mentioned above, the possibility of a third energy modality being activated due to a default condition or perhaps a third switch (not shown) disposed on the forceps 10, generator G, footswitch FS or standalone, e.g., monopolar modality.

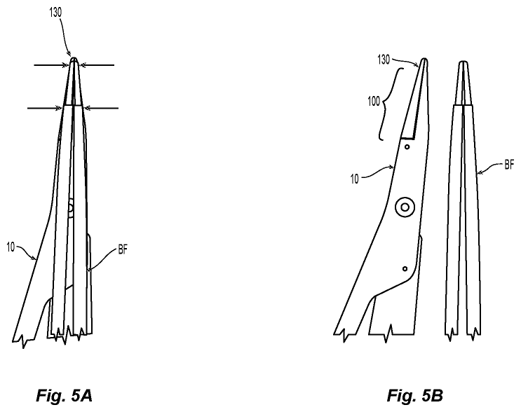

Turning to FIGS. 5A and 5B, forceps 10 is shown superimposed atop a standard bayonet forceps BF highlighting the geometry of the jaw members 110, 120 when the jaw members 110, 120 are disposed in a fully approximated position. Manufacturing the jaw members 110, 120 to mimic traditional bayonet-style forceps to both close in a tip-biased fashion and to include an end effector 100 with a tapered tip 130 allows the forceps 10 to perform precise bipolar cautery in tight spaces and around critical tissue and nerve structures with better visualization when the footswitch FS is actuated. In addition, the geometry of the jaw members 110, 120 also allows for reliable and consistent sealing of tissue utilizing the Ligasure® algorithm LS when switch 40b is actuated.

In view thereof, there is no need to switch between a Ligasure® instrument and a bipolar instrument during surgery.

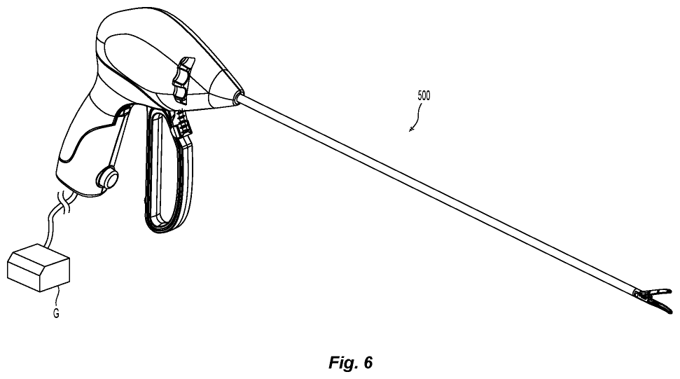

Although shown and described as a surgical system utilizing an open surgical forceps, it is envisioned that the same or similar type electrical connections may be utilized with an endoscopic forceps 500 as shown in FIG. 6. For example, one such endoscopic forceps is described in U.S. Pat. No. 10,231,776 the entire contents of which being incorporated by reference herein.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto. For example, the knife body and tube do not necessarily have to be made from the exact same materials. Similar materials, or any two materials that can be welded together to allow for a durable weld joint could be used.

What is claimed is:

1. A surgical system, comprising:
a forceps having at least one shaft configured to support an end effector assembly at a distal end thereof, the end effector including first and second opposing jaw members each including an electrically conductive plate associated therewith configured to communicate electrosurgical energy therebetween, at least one of the first or second jaw members pivotable relative to the other about a pivot such that the jaw members are selectively movable between an open position wherein the jaw members are spaced relative to one another and a closed position for grasping tissue therebetween;
an electrical generator configured to produce multiple modalities of electrical energy upon activation thereof;
a first electrical cable operably connected at one end to a first port defined in the electrical generator and at an opposite end to the forceps, the first electrical cable including electrical leads disposed therein configured to carry electrical energy to opposing electrical plates of the jaw members;
a first switch disposed on the forceps and disposed in electrical communication with at least one of the electrical leads; and
a second cable operably connected at one end to a second port defined in the electrical generator and at an opposite end to a second switch,
wherein activation of the first switch activates the electrical generator to transmit electrical energy having a first modality through the first electrical cable and to the opposing electrical plates of the jaw members and activation of the second switch activates the electrical generator to transmit electrical energy having a second modality through the first electrical cable to the opposing electrical plates of the jaw members.

2. The surgical system according to claim 1, wherein the first modality of electrical energy includes a sealing energy delivery algorithm.

3. The surgical system according to claim 1, wherein the second modality of electrical energy includes a bipolar energy.

4. The surgical system according to claim 1, wherein the first switch includes an activation switch disposed on the at least one shaft of the forceps.

5. The surgical system according to claim 4, wherein the first activation switch is an in-line activation switch that is selectively activatable when the jaw members are moved from the open position to the closed position.

6. The surgical system according to claim 1, wherein the second switch includes a footswitch remotely disposed relative to the generator.

7. The surgical system according to claim 1, wherein the first activation switch has priority over the second activation switch when activated.

8. The surgical system according to claim 1, wherein substantially simultaneous activation of the first activation switch and the second activation switch defaults the generator to deliver a third energy modality to at least one of the electrically conductive plates of the jaw members.

9. The surgical system according to claim 8, wherein the third modality of electrical energy includes a monopolar energy.

10. A forceps, comprising:
at least one shaft configured to support an end effector assembly at a distal end thereof, the end effector including first and second opposing jaw members each including an electrically conductive plate associated therewith configured to communicate electrosurgical energy therebetween, at least one of the first or second jaw members pivotable relative to the other about a pivot such that the jaw members are selectively movable between an open position wherein the jaw members are spaced relative to one another and a closed position for grasping tissue therebetween;
an electrical cable adapted to operably connect to a first port defined in an electrical generator, the electrical cable including electrical leads disposed therein configured to carry electrical energy to opposing electrical plates of the jaw members; and
a first switch disposed on the forceps and disposed in electrical communication with at least one of the electrical leads,
wherein activation of the first switch activates the electrical generator to transmit electrical energy having a first modality through the electrical cable and to the opposing electrical plates of the jaw members and activation of a second switch operably connected to and remotely disposed from the forceps activates the electrical generator to transmit electrical energy having a second modality through the electrical cable to the opposing electrical plates of the jaw members.

11. The forceps according to claim 10, wherein the first modality of electrical energy includes a sealing energy delivery algorithm.

12. The forceps according to claim 10, wherein the second modality of electrical energy includes a bipolar energy.

13. The forceps according to claim 10, wherein the first activation switch is an in-line activation switch that is selectively activatable when the jaw members are moved from the open position to the closed position.

14. The forceps according to claim 10, wherein the jaw members are tapered from a proximal end thereof to a distal end thereof.

15. The forceps according to claim 14, wherein, when disposed in the closed position, the distal ends of each jaw member combine to form a low profile tip to facilitate fine tissue dissection and cautery.
